(19) United States
(12) Patent Application Publication
(10) Pub. No.: US 2020/0395127 A1
(43) Pub. Date: Dec. 17, 2020

(54) CONNECTED SYSTEM FOR INFORMATION-ENHANCED TEST RESULTS

(71) Applicant: University of Washington, Seattle, WA (US)

(72) Inventors: Barry R. Lutz, Seattle, WA (US); Rahil Jain, Seattle, WA (US); Blythe Adamson, Seattle, WA (US)

(73) Assignee: University of Washington, Seattle, WA (US)

(21) Appl. No.: 16/764,113

(22) PCT Filed: Nov. 19, 2018
(86) PCT No.: PCT/US2018/061806

§ 371 Date: May 14, 2020

Related U.S. Application Data
(60) Provisional application No. 62/588,099, filed on Nov. 17, 2017.

(51) Int. Cl. G16H 50/70; G16H 50/20; G16H 10/60; G16H 70/60; G16H 50/80; A61B 5/00; A61B 5/0205

(52) U.S. Cl. CPC G16H 50/70; G16H 50/20; G16H 10/60; A61B 5/024; G16H 50/80; A61B 5/7275; A61B 5/02055; G16H 70/60

(57) ABSTRACT

In some embodiments, a system for using various types of information to augment the determination of a post-test probability of the presence of a condition is provided. In some embodiments, the information may include detailed health status or sub-population prevalence information, gathered in real-time, in order to provide a more accurate pre-test probability value. In some embodiments, the information may include augmentation information that helps provide a more accurate likelihood ratio for a given analytical test. In some embodiments, the more accurate likelihood ratios that are calculated using the augmentation information can be used to select a most-useful test for a given situation. In some embodiments, the augmentation information may include a multivariate model that allows a post-test probability to be determined using an analytic test result and various factors related to the subject without explicitly determining a pre-test probability nor a likelihood ratio.

### CROSS-REFERENCE(S) TO RELATED APPLICATION(S)

This application claims the benefit of Provisional Application No. 62/588,099, filed Nov. 17, 2017, the entire disclosure of which is hereby incorporated by reference herein for all purposes.

### BACKGROUND

Analytic tests, such as diagnostic tests performed in a healthcare setting, do not generally have perfect performance. Instead, some number of false positives (cases in which a condition is not actually present in a subject but the analytical test states that the condition is present) and some number of false negatives (cases in which a condition is present in a subject but the analytical test states that the condition is not present) will be generated. Accordingly, for any given analytic test, studies are normally performed to determine a sensitivity (the proportion of actual positives that are correctly identified as such) and a specificity (the proportion of actual negatives that are correctly identified as such) of the test before it is used.

Since a typical analytical test does not provide 100% certainty regarding the presence or absence of a condition, it has been desired to translate an analytical test result into a value that more intuitively represents the likelihood that a subject has a condition based on the result of the analytical test and the specificity/sensitivity of the test. One common technique used for such a translation is represented by a nomogram.

FIG. 1 is an example of a nomogram that is traditionally used for determination of a post-test probability of whether a subject is experiencing a condition given a result of an analytic test. A pre-test probability is found on the left side of the nomogram. Given the test result, a positive likelihood ratio and a negative likelihood ratio are determined based on the sensitivity and the specificity of the analytical test. The line located in the middle of the nomogram identifies the likelihood ratios. A line is then drawn through the pre-test probability and the appropriate likelihood ratio for the outcome of the analytic test to find the post-test probability on the right side of the nomogram. As shown, starting at a pre-test probability of 10%, and assuming that a positive likelihood ratio of an analytic test is 20/1, a first line is drawn from the 10% pre-test probability through the 20/1 likelihood ratio to find a post-test probability of about 70% for a positive test result. Likewise, starting at the same pre-test probability of 10%, and assuming that a negative likelihood ratio of the analytic test is 1/2, a second line is drawn from the 10% pre-test probability through the 1/2 likelihood ratio to find a post-test probability of about 5% for a negative test result. The depiction of a nomogram and the drawing of a line are an example embodiment that helps to illustrate the calculations. In some embodiments, the likelihood ratio and post-test probability may be calculated without using the actual nomogram.

While the use of a nomogram (and the underlying mathematical relationship) does help to translate from a pre-test probability to a post-test probability, there are numerous drawbacks to using this technique. For example, there is currently no consistent or accurate way to determine a pre-test probability. Even if the results of other tests are used to determine the pre-test probability, at some point there will be a test for which the pre-test probability is uncertain, and so more tests will need to be performed than would otherwise be necessary to achieve a desired probability level. As another example, the likelihood ratio used in the techniques described above is static, and is based on the experimentally determined sensitivity and specificity values for each test. This ignores many factors that can alter the likelihood ratio for a given subject or in a given situation, and cannot be updated over time in response to the collection of additional information.

What is desired are techniques that would use additional information to augment the determination of likelihood ratios, thereby improving the determination of post-test probabilities and increasing the efficacy of existing analytical tests.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In some embodiments, a method of improving accuracy of a healthcare diagnostic test is provided. A computing device receives an individual test result of an analytic test performed on a subject. The computing device determines a pre-test probability of a condition for the subject. The computing device determines a likelihood ratio of the analytic test. The computing device determines augmentation information that affects the likelihood ratio of the analytic test. The computing device generates an adjusted likelihood ratio using the augmentation information. The computing device determines a post-test probability of the condition based on the pre-test probability and the adjusted likelihood ratio. The computing device presents the post-test probability on a display device.

In some embodiments, a method is provided. A computing device determines a pre-test probability of a condition for a subject. The computing device determines a likelihood ratio of a first analytic test. The computing device determines augmentation information that affects the likelihood ratio of the first analytic test. The computing device generates an adjusted likelihood ratio using the augmentation information. The computing device determines a potential post-test probability of the condition based on the pre-test probability, the adjusted likelihood ratio, and an assumption that an individual test result of the first analytic test would indicate the presence of the condition. In response to determining that the potential post-test probability is greater than a threshold probability, the computing device presents an instruction to perform the first analytic test.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is an example of a nomogram that is traditionally used for determination of a post-test probability of whether a subject is experiencing a condition given a result of an analytic test;

FIG. 2 is a block diagram that illustrates an example embodiment of a system for augmenting the accuracy of an analytic test according to various aspects of the present disclosure;

FIGS. 3A-3B are a flowchart that illustrates an example embodiment of a method of performing an intervention for a condition for a subject according to various aspects of the present disclosure;

FIG. 4 is a flowchart that illustrates an example embodiment of a procedure for determining an analytic test to perform according to various aspects of the present disclosure; and

FIG. 5 is a block diagram that illustrates aspects of an exemplary computing device appropriate for use with embodiments of the present disclosure.

### DETAILED DESCRIPTION

In some embodiments of the present disclosure, a system for using various types of information to augment the determination of a post-test probability of the presence of a condition is provided. In some embodiments, the information may include detailed health status or sub-population prevalence information, gathered in real-time, in order to provide a more accurate pre-test probability value. In some embodiments, the information may include augmentation information that helps provide a more accurate likelihood ratio for a given analytical test, thereby allowing for a post-test probability that more accurately reflects reality without requiring changes to the analytical test itself, and without requiring detailed studies to determine the more accurate post-test probability. In some embodiments, the more accurate likelihood ratios that are calculated using the augmentation information can be used to select a most-useful test for a given situation, thus reducing the number of tests that need to be performed to obtain a clinically useful result. In some embodiments, the augmentation information may include a multivariate model that allows a post-test probability to be determined using an analytic test result and various factors related to the subject without explicitly determining a pre-test probability nor a likelihood ratio.

Many factors can affect the ability of a test to detect disease, and these effects may be manifested by an effect on the test likelihood ratio. Some effects include biological factors that affect the concentration of a target analyte in a particular sample type, components in the sample from medicines, food, or other products that affect (interfere or enhance) the biochemistry of the test, the environmental conditions under which the test is stored or performed, or differences among user types and skill levels. These effects may often be hidden from view and can interact in unforeseen or untested ways to affect test performance, but the effects can be revealed by the multivariate modeling or machine learning described herein. Further, the augmentation techniques described herein can include these factors as inputs to calculate post-test probability for a given test and patient-specific factors or other factors described herein.

In some cases these effects are related to differences in the amount of the target analyte (e.g., protein, DNA or RNA, antibody, small molecule) that is present in the sample, which may vary depending on patient factors or type of sample collected. For example, for influenza detection from nasal swabs, one commercial test is reported to have a sensitivity of 96% when tested in children but a sensitivity of 66% when tested in adults. The reason for higher sensitivity in children is that children shed more virus than adults, so there is more target analyte available for the test to detect. Thus, this is a true biological difference that affects the ability of the test to detect the disease condition. In addition, for many infectious diseases, the sensitivity of a test will depend on the stage and severity of disease. For example, the pathogen “load” (concentration) may be highest during the acute stage of infection, which may be indicated by the presence of fever or recent presence of fever or other symptoms. Severity of disease may be measured by the number or type of symptoms or the severity of one or more symptoms (e.g., body temperature). The augmentation procedure described here can include these factors as inputs to calculate post-test probability for a given test and patient-specific factors or other factors described here.

In addition, samples taken from different locations or via different methods can affect the test performance due to differences in target analyte concentration and/or components that affect biochemistry of the test. For example, swab samples taken from different locations in the nasal passage may result in different amounts of target analyte on the swab (e.g., nasal swabs taken in the nostril are comfortable for most patients, while deep nasopharyngeal samples are very uncomfortable but may provide more analyte for better test sensitivity). Similarly, some sexually transmitted infections can be diagnosed from swab samples of the genital (e.g., urethral swab, vaginal swab) or by a urine sample. However, the concentration of target analyte is typically lower than the swab samples. Further, the concentration of target analyte in urine can vary depending on the time since last urination (“first void” urine used in some cases due to higher concentration) or the portion of the urine collected (e.g., “first catch” versus “mid-stream catch” will include different amounts of organisms from the urinary tract versus the bladder, which may affect either sensitivity or specificity). In some cases there may be multiple options for the sample type that can be collected, with each sample having a different sensitivity and specificity for a given test. For tuberculosis diagnosis, sensitivity for sputum smear microscopy or sputum DNA test is poor for HIV patients, whereas a protein immunoassay for LAM in urine has good sensitivity for HIV patients but poor sensitivity for patients without HIV. Thus, the sample type can be used as an augmentation parameter. Embodiments of the present disclosure can calculate the expected performance (post-test probability) for multiple sample types to provide a comparison of the performance of different samples types for the user to select, or to recommend the best sample type or the least invasive sample type that will result in an actionable performance. In addition, the tools or methods of sample collection can affect test performance. For example, different swab types often capture or release target analytes with different efficiency, and a nasal wash often has different performance compared to a swab. The augmentation procedure described here can include these factors as inputs to calculate post-test probability for a given test and patient-specific factors or other factors described here.

The test performance can also be affected by components present in the sample from medicines, food, or other products that affect (interfere or enhance) the biochemistry of the test or measurement of the result. For example, medications used by the patient may interfere with test biochemistry or reduce pathogen load, which could change sensitivity or specificity of the test. Medications may include systemic antibiotics, antivirals, chemotherapy, immunotherapy, radiation therapy or may include topical medicines such as nasal spray, antibacterial cream, or ointments. Similarly, non-medicinal components may affect the test performance. For example, foods or food supplements (vitamins, herbs) or topical items (e.g., lotion) can contain components that affect test biochemistry. In some cases byproducts of these components can cause false positive results in tests due to similar reactivity to the intended analyte (e.g., smoking results in false positive detection of a drug adherence test for isoniazid due to similar structure of nicotine and drug metabolites). Interfering components can come from the patient, such as blood in urine or a swab sample. The augmentation procedure described here can include these factors as inputs to calculate post-test probability for a given test and patient-specific factors or other factors described here.

The presence of other health conditions may impact the test performance. For example, some diseases may be easier or harder to detect in HIV patients due to compromised immune system. Thus, retrieving health information or test results from an electronic health record or by querying the patient can provide information on other health conditions that can be used as input to calculate post-test probability for a given test.

The performance of the test can be affected by variations in the target pathogen or analyte that can vary in time and location. For example, influenza A and influenza B both infect humans and commonly circulate during flu season, but with varied prevalence across space and time, and tests often have different sensitivity and specificity for the two types. Thus, the prevalence of each type can be used as an input to calculate post-test probability as described here (through its direct effect on probability and its effect on test performance). Since the target analytes (e.g., proteins, DNA or RNA) used to detect pathogens (e.g., viruses and bacteria) can evolve over time and space, the parameters related to test performance can be updated over time or affected by location in the calculation described here (especially if a gold standard test is used to assess accuracy of the local test). This system provides an indication of the evolving test performance and can be an indicator that a test should be updated to increase performance.

Device characteristics, operating conditions, or user skill can affect the test performance. For example, tests operated at different conditions (e.g., humidity, temperature) may have different performance. Tests operated by different users (e.g., doctor, pharmacist, patient) or by users with different skill or experience levels (e.g., education level, experience with the specific test), or similar factors may affect test performance via variations in the quality of the user's test procedure or via the user's ability to read the test results (e.g., a user with poor eye sight, operation in a location with poor lighting). If a user's own device (e.g., smart phone) is used to read the test result, the phone model can affect the test performance. The test performance can also be affected by variations in the test procedure, such as too much sample or too little sample or by too long or short wait time between steps. The experience level of the user can affect test results; previous tests performed by the user can be a measure of the skill of the user in operating the test as determined from records of previous tests or self-reported experience of previous tests. Test performance may be affected by time since manufacture, closeness to expiration date, or storage condition history. The augmentation procedure described here can include these factors as inputs to calculate post-test probability for a given test and patient-specific factors or other factors described here.

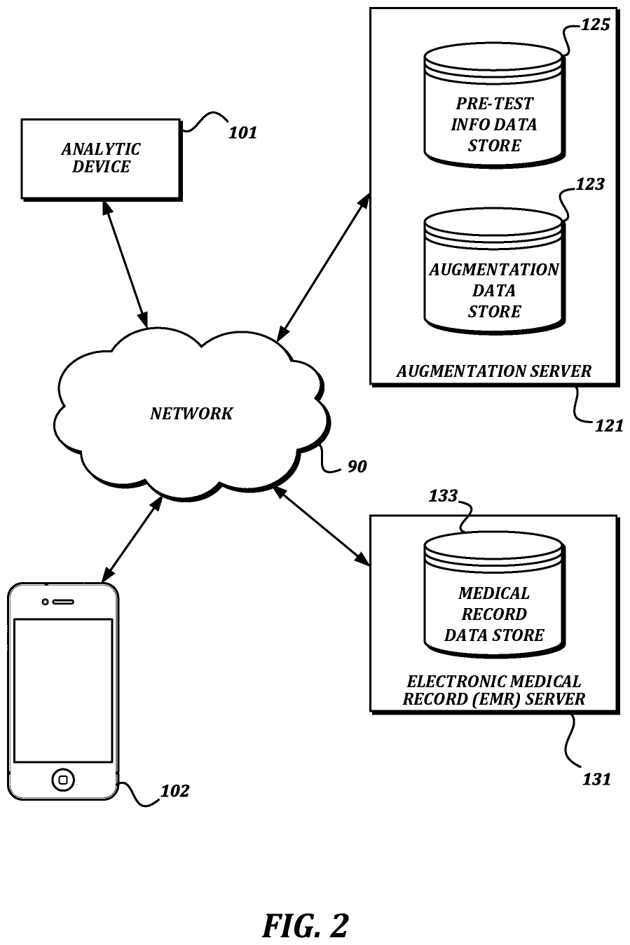

FIG. 2 is a block diagram that illustrates an example embodiment of a system for augmenting the accuracy of an analytic test according to various aspects of the present disclosure. As illustrated, the system includes an analytic device 101, a diagnostic computing device 102, an augmentation server 121, and an electronic medical record (EMR) server 131. In some embodiments, the devices 101, 102, 121, 131 are configured to communicate with each other via a network 90. The network 90 utilizes any suitable wired or wireless communication technology to enable communication between the devices 101, 102, 121, 131, including but not limited to Ethernet, USB, Firewire, Wi-Fi, 2G, 3G, 4G, LTE, Wi-MAX, Bluetooth, and the Internet.

In some embodiments, the analytic device 101 is a device that includes one or more sensors to detect a condition in a subject, and returns a result. In some embodiments, the analytic device 101 is capable of accepting a subject biological sample, performing a biochemical assay, and generating an analytical result representative of a physical condition of the subject. The analytic device 101 may then produce a test result representative of that physical condition. In some embodiments, the analytic device 101 may include a display that presents the test result to a user. In some embodiments, the analytic device 101 may transmit the test result to other components of the system via a network 90.

In some embodiments, the analytic device 101 may include one or more sensors including but not limited to optical and electrical sensors that generate data from which the analytical result may be determined. In some embodiments, the test result may be a binary result (such as yes/no, or presence/absence of a condition). In some embodiments, the test result may be a category (including but not limited to a low/medium/high value for a given property). In some embodiments, the test result may be continuous (including but not limited to a temperature, or a concentration of a particular substance).

A biochemical test is described as an example of a test performed by an analytic device 101. Examples of biochemical tests include tests for infectious diseases (e.g., protein tests, serology/antibody tests, nucleic acid tests, microscopy, small molecule tests; for diseases such as sexually transmitted infections (HIV, chlamydia, gonorrhea, syphilis, HPV, cytomegalovirus, herpes, etc.), respiratory diseases (e.g., influenza, pneumonia, pertussis, strep throat, etc.), diarrheal diseases, urinary tract infections, skin infections, etc.), pregnancy, ovulation, drugs-of-abuse, cholesterol, vitamin levels, cancer detection, diabetes onset and progression (e.g., hemoglobin A1c), sepsis, stress markers, inflammation, blood glucose, and other tests that detect analytes from samples taken from the body. Similar biochemical tests may be used for veterinary care, agricultural disease/health testing, and water quality. For example, biochemical tests can be used for detection of environmental conditions from deployed sensors (e.g., biothreats, water quality), diagnosis of agricultural conditions (e.g., pest infestation, hydration deficiencies, plant disease, soil conditions), diagnosis of animal health conditions, testing for food spoilage, identification of genetically-modified plants and animals, testing for banned substances or trade items, and many other health and non-health applications.

Though a biochemical test is mentioned above as an example of a test performed by an analytic device 101, in some embodiments, other types of analytic tests, such as other types of diagnostic tests, other types of health assessment devices, and other types of non-health measurement devices may be used. Non-limiting examples include various modes of imaging (e.g., magnetic resonance imaging, CT, X-ray, ultrasound imaging, optical imaging, fluorescence, radiography, nuclear imaging, elastography, tactile imaging, photoacoustic imaging, thermography, tomography, echocardiography, near-infrared spectroscopy) and other means of detecting biological states (e.g., cardiac electrical signal, cardiac pulse, respiratory patterns, measurement of physical motion (trembling, sleeping movements), sound (snoring, wheezing, coughing), measurements of strength or coordination, blood pressure, body temperature, blood oxygen levels, sweat production, spirometry, lung function testing). Further, the analytic device 101 may determine a non-health measurement, such as a measurement of structural integrity (defects in airplane parts, bridges, pipelines, storage tanks), detection of pollutants and water quality, presence of radioactive substances, human motion from a cell phone (e.g., to determine likelihood that the human is involved in a particular task), detection of environmental conditions from deployed sensors (e.g., biothreats, pollution, contamination, water quality), diagnosis of agricultural conditions (e.g., pest infestation, hydration deficiencies, plant disease, soil conditions).

Though a single analytic device 101 is illustrated, in some embodiments, multiple analytic devices 101 may be present and used within the system.

In some embodiments, the EMR server 131 is one or more server computing devices that store and manage electronic medical records. The EMR server 131 may store the electronic medical records in a medical record data store 133. In some embodiments, the electronic medical records store basic information about patients, including but not limited to age, sex, gender, height, and weight. In some embodiments, the electronic medical records may store past test results produced by various analytic devices 101 and/or information regarding past or current health conditions, symptoms, and/or diagnoses. In some embodiments, the electronic medical records may store information about treatments provided to patients, including but not limited to medications being administered and procedures that have been performed. In some embodiments, the EMR server 131 may receive and respond to queries via the network 90 from other components of the system for information about a given patient, and may receive information via the network 90 from other components of the system to be stored in the electronic medical records. In some embodiments, the EMR server 131 may be accessible by a computer terminal, desktop computer, mobile computing device, or any other type of computing device, and electronic medical records may be browsed using the computing devices. In such embodiments, test results from the analytic devices 101 and other information may be entered into the electronic medical records using the computing devices.

In some embodiments, the augmentation server 121 is one or more server computing devices that provide information that can be used to augment or otherwise improve the determination of a post-test probability for a test performed by an analytic device 101. In some embodiments, the augmentation server 121 may provide the information directly to the diagnostic computing device 102 via the network 90. In some embodiments, the augmentation server 121 may be accessible via a user interface including but not limited to a computer terminal or a web-based interface. In such embodiments, a user may use the user interface to query the augmentation server 121 for information, and may then manually enter the information into the diagnostic computing device 102.

As shown, the augmentation server 121 may use a pre-test information data store 125 and an augmentation data store 123 to store such information. The pre-test information data store 125 may include information relevant to determining a pre-test probability for a condition for a given subject at a given location and time. This information may include, but is not limited to: prevalence of the condition in an overall population, prevalence of the condition based on a time of year, prevalence of the condition based on sub-population characteristics (e.g., high-risk or low-risk subpopulations determined by a characteristic of the subject), and a prevalence of the condition within a geographical area associated with the subject. The augmentation data store 123 may include information relevant to determining and/or adjusting a likelihood ratio for an analytic test, including but not limited to a sensitivity and/or specificity for the analytic test, one or more likelihood ratios for the analytic test, effects that various factors have on the efficacy of the analytic test, or models that can be used to determine a likelihood ratio or post-test probability based on various factors.

In some embodiments, the diagnostic computing device 102 receives test results generated by the analytic device 101, receives information stored by the augmentation server 121 and/or the EMR server 131, and uses the received information to determine a post-test probability that a subject has a condition. In some embodiments, the diagnostic computing device 102 is a mobile computing device such as a smartphone or a tablet computing device. In some embodiments, the diagnostic computing device 102 is a desktop computing device. In some embodiments, the functionality of the diagnostic computing device 102 may be provided by a server computing device, which is accessed by a user using a mobile computing device or a desktop computing device.

The separation of components in the block diagram provided in FIG. 2 is an example only. In some embodiments, some components illustrated as single components in FIG. 2 may be split into multiple components. In some embodiments, some components illustrated as separate components in FIG. 2 may be combined into a single component. For example, in some embodiments, the analytic device 101 may perform some or all of the functionality described as being performed by the diagnostic computing device 102. As another example, in some embodiments, at least some of the functionality of the diagnostic computing device 102—particularly that relates to the use of augmentation information to determine likelihood ratios and/or post-test probability—may be provided by the augmentation server 121 as a server-based or cloud-based service. In such embodiments, the diagnostic computing device 102 as illustrated may be used to present a user interface to the user and to collect information from the user, but the determination of a post-test probability and other computations may be performed by a server.

One non-limiting example embodiment of the present disclosure includes an over-the-counter pregnancy test that supports at least some functionality of both the diagnostic computing device 102 and the analytic device 101 described herein. The test device could contain a light and photodiode to detect the presence or absence of a colored test line. The combined unit may be included in a plastic case, and the entire unit including the detector, electronics, and battery may be discarded after use. The unit may solicit input from the subject such as time since last menstrual cycle, body temperature, and age. The unit may also include communication functionality. The user's smartphone may be used to download an app, and the smartphone may connect to the communication functionality of the unit via Bluetooth or other communication technique to allow input of patient information and to display the results. In this way, many functions of the diagnostic computing device 102 and the analytic device 101 are provided by the unit, and the smartphone is used only as an interface. The unit may also determine the location of the device and the local time of day. This information can be used to calculate the likelihood of pregnancy as described in this disclosure, with calculation performed either remotely on a server or locally on the device, and the result can be presented to the user as a probability or a categorical result based on threshold probabilities.

As another example, the functionality of the analytic device 101, the diagnostic computing device 102, and the augmentation server 121 may be designed as a single, autonomous unit that operates without dependence on any other device. The unit can be designed with user interface devices such as a screen display with buttons to input values, or a set of fixed buttons or switches that are moved to positions indicate each patient factor, and the device can be designed to provide output via the screen display or other indicators such as light-emitting diodes (LEDs). The device could include stored data for the augmentation calculation, and could operate without a requirement to connect to a remote source. In this case the device could perform all functions of the augmentation locally and independently without any other device or connectivity. A similar device could be autonomous in all functions except that this connects to a network using onboard communication to send or receive data.

A “data store” as described herein may include any suitable device configured to store data for access by a computing device. One example of a data store is a highly reliable, high-speed relational database management system (DBMS) executing on one or more computing devices and accessible over a high-speed network. Another example of a data store is a key-value store. However, any other suitable storage technique and/or device capable of quickly and reliably providing the stored data in response to queries may be used, and the computing device may be accessible locally instead of over a network, or may be provided as a cloud-based service. A data store may also include data stored in an organized manner, such as files in a file system, on a computer-readable storage medium, as described further below. One of ordinary skill in the art will recognize that separate data stores described herein may be combined into a single data store, and/or a single data store described herein may be separated into multiple data stores, without departing from the scope of the present disclosure.

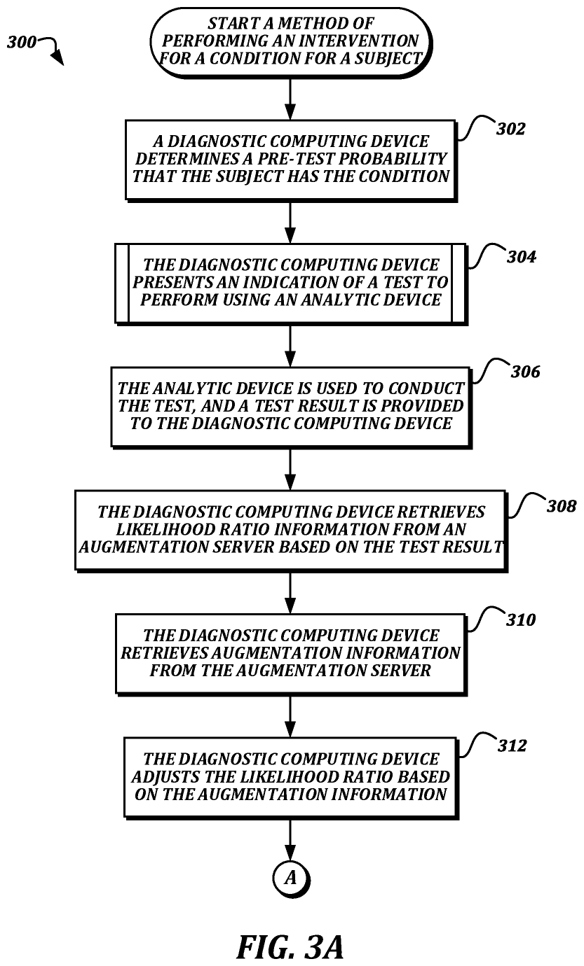

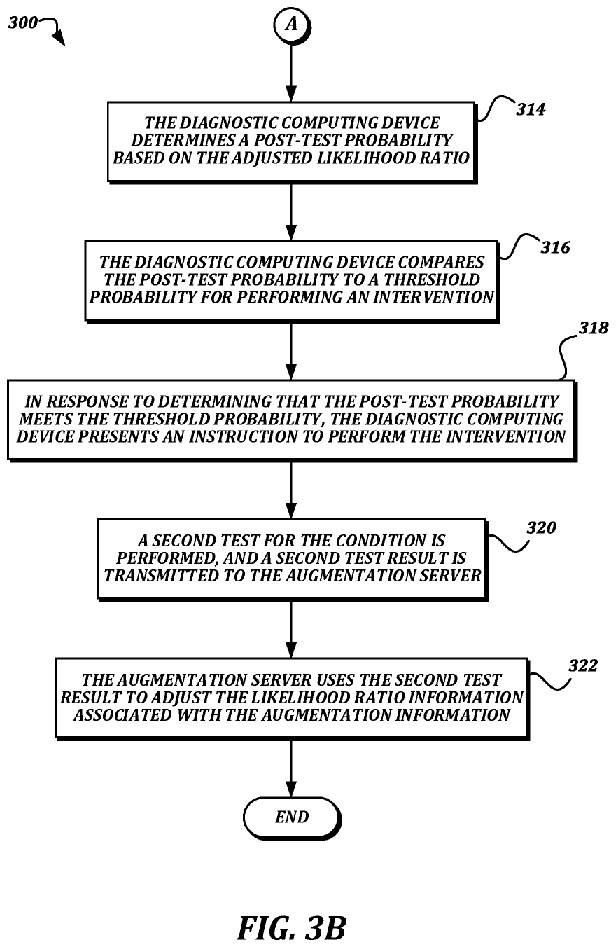

FIGS. 3A-3B are a flowchart that illustrates an example embodiment of a method of performing an intervention for a condition for a subject according to various aspects of the present disclosure. As discussed above, the condition may be a health condition including but not limited to a disease, an injury, a medical condition, or a presence of a drug. In some embodiments, the condition may be a non-health condition, including but not limited to structural integrity, detection of pollutants, and detection of environmental conditions.

From a start block, the method 300 proceeds to block 302, where a diagnostic computing device 102 determines a pre-test probability that the subject has the condition. Embodiments of the present disclosure, which collect information from multiple sources to compute a pre-test probability, can generate more accurate pre-test probability values than traditional methods, at least because many conditions are affected by many such factors. Using data from a public vaccination dataset (IVMC 2007) that included 21,311 adult patients, we developed a multivariate model that identified patient-specific factors that affected the prevalence of having influenza. For example, we found that both vaccination status and family health affects the pre-test probability of influenza in adults:

| Characteristic | Prevalence |
|---|---|
| General Population | 14.2% |
| Family w/ Children Age 5+, school-going | 15.5% |
| Child vaccinated for flue | 13.0% |
| Child has ILI symptoms | 21.2% |

Patient-specific factors, time, and location are expected to affect the patient-specific prevalence for other conditions. For example, the prevalence for influenza is expected to be affected by time, location, symptoms (e.g., fever, cough, sore throat) and social factors (e.g., vaccination status, family size and ages); malaria is expected to be affected by time, location, symptoms (e.g., fever); urinary tract infection prevalence is expected to be affected by symptoms (e.g., fever, discoloration of urine) and behavioral risks (e.g., sexual activity); HIV is expected to be affected by location (living in a high-prevalence area), and behavior (e.g., risky sexual behavior), and social factors (e.g., socioeconomic status), as well as other factors such as history or current infection with a different sexually-transmitted disease; strep throat prevalence is expected be affected by time, symptoms (e.g., fever, swollen tonsils, absence of cough), and social factors (e.g., family size and ages, exposure risk). These patient factors can be provided to the system either via input by the user (e.g., patient, doctor, pharmacist, healthcare worker, teacher, parent, caregiver), via retrieval from a server (e.g., EHR server, Augmentation Server), or via a combination of these sources.

| | Temporal | Geographical | Symptomatic | Behavioral | Social Factors |
|---|---|---|---|---|---|
| | (e.g., | (e.g. city, | (e.g. fever, | (e.g., sexual | (e.g. vaccination, |
| | incidence) | county) | cold) | activity) | sick kid) |
| Flu | X | | X | | X |
| Malaria | X | X | X | | |
| UTI | | | X | X | |
| HIV | | X | | X | |
| Strep | X | | X | | X |

The presence of other health conditions may impact the pre-test probability, or more broadly affect the augmentation parameters used to calculate post-test probability. Some diseases share symptoms, and presence of one condition may reduce the probability that a second condition is present. For example, a patient with fever may have increased probability of having influenza and strep throat, but a confirmed diagnosis of strep throat may reduce the probability that the patient has influenza. In addition, while symptoms or other patient factors described here are largely focused on those that increase probability of a given condition, symptoms or other patient factors can also have a negative contribution to probability that the patient has a given condition. For example, in a patient with fever, the presence of cough reduces the probability that the patient has strep throat. Thus, calculation of post-test probability can include factors that increase or decrease probability that the condition under consideration is present. In addition, the presence of a condition that shares risk factors (e.g., for infectious disease, the same transmission route) with the condition under consideration can affect the probability of that condition. For example, the presence of any sexually-transmitted infection (e.g., Gonorrhea) can increase the probability that the patient has a different sexually-transmitted infection (e.g., Chlamydia) since it is an indication of a shared risk factor in the patient (e.g., an indication of unsafe sex, the shared transmission route). The presence of one condition can also affect the probability of having another condition by changing the susceptibility of the patient to acquiring the second condition through biological changes. For example, a person with HIV or undergoing cancer treatment may have a compromised immune system that makes them more susceptible to an infection such as pneumonia. Thus, retrieving health information or tests results from the EMR server 131 or by receiving the information from the patient can provide information on other health conditions that can be used as input to calculate pre-test probability and/or to calculate post-test probability for a given test.

In some embodiments, the diagnostic computing device 102 could determine the pre-test probability by retrieving prevalence information for the population as a whole (or from a sub-population associated with a characteristic of the subject or a geographical area associated with the subject) from the pre-test information data store 125. In some embodiments in which the diagnostic computing device 102 is co-located with the subject, a GPS location of the diagnostic computing device 102 may be used to determine the appropriate geographical area to use for the sub-population prevalence information. In some embodiments, the diagnostic computing device 102 could determine the pre-test probability by receiving a post-test probability determined by some other analytic test or sequence of tests (such as a test for a reported symptom or combination of symptoms (e.g., presence of cough, presence of fever)), and could determine the pre-test probability as the post-test probability of having the condition given the symptoms. Such information may be stored in the medical record data store 133 by the EMR server 131. In some embodiments, other information stored in the medical record data store 133, including but not limited to family illness status, family history, and vaccination status, may be used to further adjust the pre-test probability.

In some embodiments, this information for determining the pre-test probability could be retrieved by the diagnostic computing device 102 directly from augmentation server 121 and/or the EMR server 131 via the network 90. In some embodiments, this information for determining the pre-test probability could be retrieved from the augmentation server 121 and/or the EMR server 131 by a user, and input into a user interface of the diagnostic computing device 102 by the user. In some embodiments, instead of or in addition to using information stored in the augmentation server 121 and/or the EMR server 131, information for determining the pre-test probability may be entered directly into the diagnostic computing device 102. For example, in some embodiments, the diagnostic computing device 102 may present one or more questions to a subject to collect information regarding factors such as reported symptoms, exposure risk, vaccination status, age, or any other type of factor associated with the subject, without such information being retrieved from the EMR server 131 or the augmentation server 121. In some embodiments, the user may use other techniques to determine the pre-test probability, and may directly enter the pre-test probability into the user interface of the diagnostic computing device 102.

The use of information from the augmentation server 121 and/or the EMR server 131 provides various technical improvements to the determination of the post-test probability. For example, using this information, which can be updated on the servers constantly and can provide data that is accurate at any given time, helps the system determine consistent and accurate pre-test probability information. Having this accurate pre-test probability improves the accuracy of the post-test probability determination further than would be possible using traditional techniques to determine the pre-test probability that do not use such information.

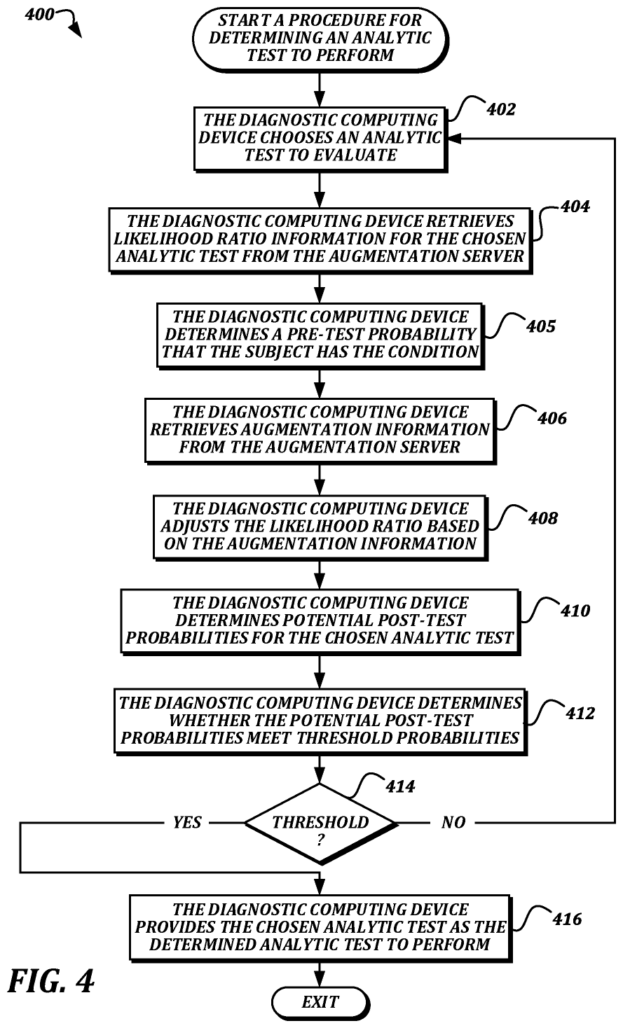

Next, the method 300 advances to a procedure block 304, where a procedure is executed wherein the diagnostic computing device 102 presents an indication of a test to perform using an analytic device 101. The indication may include an identification of the analytic device 101 or an identification of a type of analytic device 101 to use, particularly if more than one type of analytic device 101 is present in the system. In some embodiments, this indication is merely presented in order to help provide guidance to the user. However, in some embodiments, a procedure (such as the procedure illustrated in FIG. 4) may be executed to choose a test to perform based on determined possible outcomes of various tests on the subject being tested. Such a procedure may be used in order to avoid performing a test that would not provide a clinically conclusive result, and may thus help to avoid wasted time and resources. In some embodiments, the procedure block 304 may be optional, in that the user may already have determined what test should be performed before the method 300 begins, or only a single type of test may be available.

At block 306, the analytic device 101 is used to conduct the test, and a test result is provided to the diagnostic computing device 102. An appropriate procedure for conducting the test using the analytic device 101 is used. For example, if the analytic device 101 is a biological testing device such as an influenza testing device, a swab sample may be collected from the subject, and provided to the analytic device 101. The analytic device 101 may then perform an assay, and determine an analytical result indicative of the presence of (or proportional to the concentration of an antigen or other analyte related to) influenza. In some embodiments, this analytical result may be presented as a test result on a display of the analytic device 101, and the test result may be entered into a user interface of the diagnostic computing device 102. In some embodiments, the diagnostic computing device 102 may include a camera, light detector, fluorescence detector, electrical detector, or magnetic detector that allows the diagnostic computing device 102 to read the analytical result presented by the analytic device 101. In some embodiments, the analytic device 101 may electronically provide the test result to the diagnostic computing device 102 via the network 90. In some embodiments, the test result may be stored in the medical record data store 133, and the diagnostic computing device 102 may retrieve the test result electronically from the EMR server 131 via the network 90.

At block 308, the diagnostic computing device 102 retrieves likelihood ratio information from an augmentation server 121 based on the test result. In some embodiments, the likelihood ratio information is the likelihood ratio itself, while in some embodiments, the likelihood ratio information is other information (such as sensitivity and specificity) that can be used to determine the likelihood ratio. In some embodiments, the diagnostic computing device 102 may use an identity of the test performed in order to retrieve a likelihood ratio stored by the augmentation server 121 for the test. In some embodiments, separate likelihood ratios may be stored by the augmentation server 121 for separate test outcomes. For example, the augmentation server 121 may store a positive likelihood ratio that represents the likelihood ratio to be used when the test indicates that the condition is present, and a negative likelihood ratio that represents the likelihood ratio to be used when the test indicates that the condition is absent. In such an embodiment, the diagnostic computing device 102 may retrieve the appropriate likelihood ratio depending on the test outcome. In some embodiments, the augmentation server 121 may store sensitivity and specificity information for the test, and may provide that information to the diagnostic computing device 102 so that the diagnostic computing device 102 may compute its own likelihood ratio. In some embodiments, the augmentation server may store true positive, true negative, false positive, and false negative test information that may be retrieved by the diagnostic computing device 102 to compute sensitivity, specificity, and/or likelihood ratios.

At block 310, the diagnostic computing device 102 retrieves augmentation information from the augmentation server 121. In some embodiments, the augmentation information includes effects that various factors have on the efficacy of the analytic test. Some examples of factors for which augmentation information may indicate an effect for some analytic tests include, but are not limited to: patient age, demographics, geographical location, date and time, body temperature, heart rate, respiratory rate, medication status, vaccination status, expertise level of the test administrator, type of analytic device 101 used, success in previously performing the test, expiration date of the test, and ability of the test administrator to read the test results. In some embodiments, the augmentation information includes a known effect of the information on the sensitivity and/or specificity. For example, it may be determined experimentally that a given test has a first sensitivity and/or specificity in the pediatric population and a second sensitivity and/or specificity in the adult population. Accordingly, the augmentation information may include adjustments to be made to the sensitivity and/or specificity based on age. In some embodiments, the effect on sensitivity and/or specificity of a given piece of augmentation information (or for a given combination of augmentation information) may not initially be known, but may be determined by the system over time as discussed further below. At block 312, the diagnostic computing device 102 adjusts the likelihood ratio based on the augmentation information. As noted above, in some embodiments, this calculation may be performed by the augmentation server 121 instead of the diagnostic computing device 102. In some embodiments, the augmentation information includes an effect that a given factor would have on the sensitivity/specificity (e.g., higher sensitivity for children, lower sensitivity for adults), and information stored and provided by the EMR server 131 (or input by the user to the diagnostic computing device 102) may provide the value of the factor for a given subject (e.g., this test was run on a child). In some such embodiments, the diagnostic computing device 102 may receive the factor values and adjust the likelihood ratio as indicated by the augmentation information from the augmentation server 121. In other such embodiments, the diagnostic computing device may receive the factor values and provide them to the augmentation server 121 for adjusting the likelihood ratio as indicated by the augmentation information.

As a non-limiting example, influenza rapid diagnostic tests (RDTs) based on immunoassay detection of influenza proteins are more sensitive when tested in children than when tested in adults. One manufacturer's test is reported to have 96% sensitivity when tested on children and 66% sensitivity when tested in adults. The reason for higher sensitivity in children for this test is that children shed more virus than adults, so there is more analyte available for the test to detect. Thus, this is a true biological difference that affects the ability of the test to detect the disease condition. The augmentation information can represent how age affects sensitivity, and the diagnostic computing device 102 can adjust the likelihood ratio based on the age factor value using this information. Other factors such as severity of symptoms, body temperature, time since onset of fever, are expected to also influence the test performance, but these factors are not measured in typical studies nor used in typical diagnosis. In addition, other factors are anticipated to affect test performance: presence of interfering substances (e.g., blood on the swab, use of nose spray by the patient), expiration date of the test, the user's ability to read the test results (e.g., glasses, cell phone model). The discussion below explains how these factors that are not measured in typical studies, and that are not studied in combination with each other, can be used by embodiments of the present disclosure. In some embodiments, the augmentation information retrieved from the augmentation server 121 may include all of the information needed by the diagnostic computing device 102 to adjust the likelihood ratio, such as a new likelihood ratio associated with a given factor. In some embodiments, the augmentation server 121 may provide a multivariate model that may be used by the diagnostic computing device 102 in order to determine the impact of multiple interrelated factors on the likelihood ratio. For example, it may be experimentally determined that subject age has a first effect on the likelihood ratio, a medication interaction has a second effect on the likelihood ratio, and a lower level of training for the test administrator (e.g., the test was self-performed by the subject instead of being performed by a nurse or doctor) has a third effect on the likelihood ratio. However, there likely would have been no experimental study performed on how these three effects interact with each other. Accordingly, a multivariate model generated by machine learning (as discussed further below) may be useful to uncover the otherwise hidden interactions between these effects and improve the efficacy of the analytic test even further.

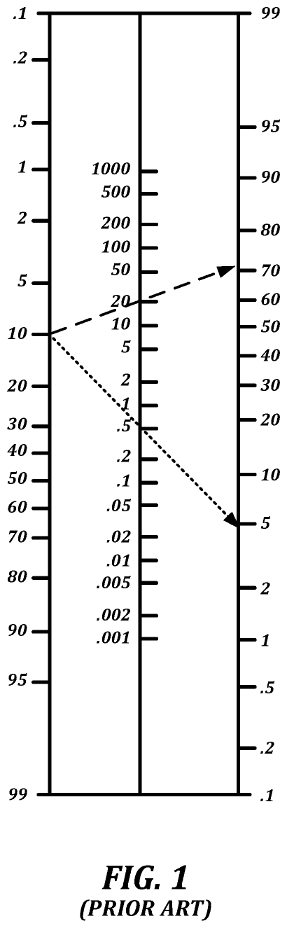

The method 300 then proceeds to a continuation terminal (“terminal A”). From terminal A (FIG. 3B), the method 300 proceeds to block 314, where the diagnostic computing device 102 determines a post-test probability based on the adjusted likelihood ratio. The appropriate adjusted likelihood ratio (e.g. either the positive likelihood ratio or the negative likelihood ratio) is chosen based on the result of the analytic test. Graphically, the post-test probability may be computed using the nomogram as discussed above with respect to FIG. 1, but with the adjusted likelihood ratio and the more detailed pre-test probability determined in the present method 300 instead of the static likelihood ratio and uncertain pre-test probability described above. In some embodiments, the diagnostic computing device 102 will instead calculate the post-test probability based on the adjusted likelihood ratio and the pre-test probability using a technique similar to the following:

$$PreTest Odds = \frac{PreTest Probability}{( 1 - PreTest Probability )}$$

$$PostTest Odds = PreTest Odds * Adjusted Likelihood Ratio$$

$$PostTest Probability = \frac{PostTest Odds}{( 1 + PostTest Odds )}$$

At block 316, the diagnostic computing device 102 compares the post-test probability to a threshold probability for performing an intervention. The intervention may be any suitable action to respond to the presence of the condition, including but not limited to performing a procedure, administering a medication, performing another test, going to an emergency room, and avoiding contact with others. In some embodiments, more than one intervention and/or more than one threshold probability may be considered. For such embodiments, a post-test probability that is greater than a first threshold probability but less than a second threshold probability may be associated with a first intervention, while a post-test probability that is greater than both the first threshold probability and the second threshold probability may be associated with a second intervention.

In some embodiments, the user may set the threshold probability as appropriate based on the severity of the condition, potential side effects or cost of the intervention, or other appropriate factors. For example, if the side effects and cost of the intervention are negligible, the threshold probability for taking action in response to a positive test may be relatively low. Meanwhile, if the side effects and cost of the intervention would have a large impact on the subject, then the threshold probability for taking action in response to a positive test may be relatively high. Likewise, if a condition is grave, the threshold probability for taking no action in response to a negative test may be relatively low, while if a condition is not serious, then the threshold probability for taking no action in response to a negative test may be relatively high.

In some embodiments, the threshold probabilities may be pre-programmed into the diagnostic computing device 102, may be retrieved by the diagnostic computing device 102 from the augmentation server 121 or EMR server 131, or may be obtained from another authoritative source. In some embodiments, the threshold probabilities may be automatically adjusted based on factors associated with the subject, including but not limited to age.

At block 318, in response to determining that the post-test probability meets the threshold probability, the diagnostic computing device 102 presents an instruction to perform the intervention. In some embodiments, in the case of a positive test that meets the positive probability threshold (e.g., is greater than or is greater than or equal to the probability threshold value), the instruction may present an indication of a medication to administer, a procedure to perform, or some other type of intervention.

While embodiments that include block 316 and 318 may provide technical advantages in the way of providing highly accurate, evidence-based intervention decisions, in some embodiments, the post-test probability may simply be presented to the user, such that the user can decide on an intervention themselves based on the raw post-test probability.

Block 318 assumes that the result of the test was positive for the presence of the condition. In some embodiments, in the case of a negative test that meets the negative probability threshold (e.g., is less than or is less than or equal to a negative probability threshold value), the instruction may not be presented in block 318, or an indication may be presented that the subject does not have the condition. In some embodiments, if the post-test probability does not meet the probability threshold, then an indication of an inconclusive result may be presented, along with an instruction to perform an additional test before drawing conclusions regarding the presence or absence of the condition. In some embodiments, such situations can be avoided by performing the procedure described in FIG. 4.

At this point in the method 300, the subject has been tested for the presence of the condition, and the method 300 may terminate. However, in some embodiments, the method 300 may proceed to improve the data stored by the augmentation server 121. Accordingly, at block 320, a second test for the condition is performed, and a second test result is transmitted to the augmentation server 121. The second test is another test for the condition that is known to have a high specificity and sensitivity, and can be considered a “gold standard” test for the presence or absence of the condition. Instead of a single test, the second test may include the results of multiple tests in order to increase the post-test probability to near-certainty. In some embodiments, this second test result (or combination of test results) may be assumed by the augmentation server 121 to be correct, in that if the second test result indicates the presence of the condition, it will be assumed by the augmentation server 121 that the condition was actually present in the subject, and if the second test result indicates the absence of the condition, it will be assumed by the augmentation server 121 that the condition was not present in the subject. In some embodiments, an experimentally determined sensitivity and specificity may be used to statistically adjust data generated by the second test.

At block 322, the augmentation server 121 uses the second test result to adjust the likelihood ratio information associated with the augmentation information. In some embodiments, the augmentation server 121 may use the second test result to adjust totals of true positives, true negatives, false positives, or false negatives for the analytic test that was performed in block 306. That is, if the analytic test of block 306 indicated presence of the condition but the second test result indicated absence of the condition, the number of false positives for the analytic test would be incremented, and so on. The new totals of true positives, true negatives, false positives, and false negatives may then be used to determine new sensitivity and specificity values for the analytic test. In some embodiments, the analytic test result from block 306, the second test result, the pre-test probability value, the augmentation information, and the factors retrieved from the EMR server 131 may be inputs or training data for a machine learning or multivariate model. Using the second test result as the labels for the training data, the augmentation server 121 may determine a model that represents how the factors interact to produce a likelihood ratio that is independent from the experimentally determined sensitivity and specificity for any single factor. This model may then be used as the augmentation data instead of simply using the sensitivity and specificity as described above.

In some embodiments, the information processed by the system may not just enhance the augmentation data, but may also enhance the prevalence information stored in the pre-test information data store 125. The second test result, the first test result, or both, can be used to update the prevalence information, either in association with geography or other factors specific to the subject, or for the population as a whole. In some embodiments, the first test result may be used to enhance the prevalence information, while limited sampling with the second test result can be used to apply statistical corrections to the enhancements made using the first test result.

The method 300 then proceeds to an end block and terminates.

Though embodiments are described above in which the augmentation information is used to adjust a likelihood ratio, and the adjusted likelihood ratio is then used along with a pre-test probability to determine a post-test probability, some embodiments may use less information to come to a similar result. For example, in some embodiments, the multivariate model that uses factors associated with the subject may be used with the test result alone (in other words, without determining a separate pre-test probability value) in order to directly determine the post-test probability from the test result and the factors associated with the subject. Though not limited to such embodiments, such embodiments may be particularly suited to having the functionality described as being performed by the diagnostic computing device 102 actually being performed on a server such as the augmentation server 121, so that more powerful computing resources can be used to process the multivariate model, and so that the specifics of the multivariate model can be more easily secured from unauthorized review.

FIG. 4 is a flowchart that illustrates an example embodiment of a procedure for determining an analytic test to perform according to various aspects of the present disclosure. The procedure 400 is an example of a procedure suitable for use in block 304 of FIG. 3A, though in some embodiments it may be used independently from the method 300. Multiple analytic tests may be available for the same condition, but may have different characteristics. Some characteristics, such as cost, ease of use, invasiveness, availability of materials, turnaround time, and availability of trained administration personnel, may give rise to preferences of one test over another. However, choosing a test on these characteristics alone may cause a test to be performed that does not provide a result that meets the threshold probability criteria discussed above. These inconclusive tests can be avoided by using the procedure 400 to ensure that a selected test will provide a result that meets the threshold probability criteria.

From a start block, the procedure 400 advances to block 402, where the diagnostic computing device 102 chooses an analytic test to evaluate. In some embodiments, an ordered list of analytic tests may be obtained by the diagnostic computing device 102, and the first analytic test may be chosen from the list. This list may be ordered by cost, invasiveness, ease of use, user preference, or in any other suitable order. In some embodiments, the list may be filtered further by checking inventory amounts, analytic device 101 booking availability or staffing resources, in order to only include tests that could be performed within a given time period such as an office visit.

At block 404, the diagnostic computing device 102 retrieves likelihood ratio information for the chosen analytic test from the augmentation server 121. At block 405, the diagnostic computing device 102 determines a pre-test probability that the subject has the condition. At block 406, the diagnostic computing device 102 retrieves augmentation information from the augmentation server 121. At block 408, the diagnostic computing device 102 adjusts the likelihood ratio based on the augmentation information. The actions performed in blocks 404, 405, 406, and 408 are similar to the actions performed in blocks 308, 302, 310, and 312 of FIG. 3A and described above. Accordingly, these actions are not described again here, for the sake of brevity.

At block 410, the diagnostic computing device 102 determines potential post-test probabilities for the chosen analytic test. This is similar to the actions performed in block 314 of FIG. 3B, though in block 416, both the positive post-test probability and the negative post-test probability may be determined so that both potential test outcomes may be analyzed. At block 412, the diagnostic computing device 102 determines whether the potential post-test probabilities meet threshold probabilities. In some embodiments, the diagnostic computing device 102 checks whether the positive post-test probability is greater than a positive threshold probability, and checks whether the negative post-test probability is less than a negative threshold probability, as discussed above.

The procedure 400 then advances to a decision block 414, where a determination is made regarding whether the threshold probabilities were met. If the threshold probabilities were not met, then the result of decision block 414 is NO, and the procedure 400 returns to block 402, where a different analytic test is chosen for evaluation. Otherwise, if the threshold probabilities were met, then the result of decision block 414 is YES, and the procedure 400 advances to block 416, where the diagnostic computing device 102 provides the chosen analytic test as the determined analytic test to perform. The procedure 400 then advances to an exit block and terminates.

In some embodiments, if a single analytic test cannot be determined that would satisfy both the positive post-test probability and the negative post-test probability, then a pair of tests (one that meets the positive threshold probability and one that meets the negative threshold probability) may be provided by the procedure 400. In some embodiments, if none of the analytic tests meet the threshold probabilities, the procedure 400 may provide a recommended series of tests that, if executed in sequence (e.g., the post-test probability of a first test is used as the pre-test probability of a second test), would meet the threshold probabilities while minimizing the combined unwanted test characteristics of invasiveness, cost, and so on.

Though the above procedure 400 describes analyzing tests in a predetermined order, in some embodiments, multiple tests may be analyzed in parallel in order to allow more tests to be analyzed in a given time. For example, if information for five analytic tests is present in the system, predicted post-test probabilities for all five analytic tests may be determined and presented to the user, such that the user can pick a desired analytic test based on a comparison of the predicted post-test probabilities.

In addition to the technical benefits described above, embodiments of the present disclosure can also provide improved access to healthcare, particularly for individuals for whom accessing a physician is difficult or impossible. In a non-limiting example, an individual may suspect that they have the flu. The individual downloads an app to their smartphone that provides at least some of the functionality of the diagnostic computing device 102 discussed above. The app prompts the user with questions to provide values for factors such as age, exposure risk, vaccination status, and symptoms. The app transmits the responses along with location to the augmentation server 121, which uses the responses and a real-time prevalence as input to a multivariate model to calculate a post-test probability that the individual has the flu. If the post-test probability is above a threshold value, the server returns a result to the app indicating that a test should be performed (e.g., the individual should obtain and perform an over-the-counter test). The same information may be used to prompt a prescription for the test. The individual obtains the test, runs the test, and provides the app with additional factors from the time of the execution of the test that are known to influence the test performance (e.g., time since fever, other medications). After the test is run, the app guides the individual to read the test and input the test result into the app (this could also be read by a camera, electrical measurement, or other sensing method). The app sends the test result and the additional factors to the augmentation server 121, which uses the factor information (including previously gathered information), the calculated pre-test probability, and test identity (to retrieve test-specific data) as inputs to a model that calculates post-test probability. The post-test probability is then compared to threshold probabilities to recommend an intervention for the individual. For example, if the test is negative and the post-test probability is below a negative threshold probability, it is unlikely that the subject has the condition, and so the recommendation may be to treat the symptoms with over-the-counter medications and that a visit to a physician is unnecessary. If the test is positive and the post-test probability is above a positive threshold probability, the recommendation may be to visit an urgent care clinic or other physician in order to be thoroughly examined and treated, or to simply take a predetermined medication for the condition. If the test is positive and the post-test probability is not above the positive threshold probability, the recommendation may be to visit a physician to be diagnosed, or to perform a different test to obtain a more precise result. These telehealth recommendations can help individuals who are not likely to be diagnosed with the condition to avoid time-consuming and expensive visits to the physician, and can help encourage individuals who are likely to be diagnosed with the condition or with unreliable diagnoses to seek care.

In another non-limiting example, embodiments of the present disclosure provide physicians with evidence justifying clinical testing decisions, and helping physicians avoid waste by ordering inconclusive tests. As more tests of varying efficacy/convenience are developed, such evidence is becoming increasingly useful. For example, there have historically been few options for point-of-care flu tests. Nearly all available flu tests were rapid diagnostic tests (RDTs) that are similar to a pregnancy test. As a category of devices, these RDTs often have lower sensitivity and/or specificity than many lab tests, such as PCR and other nucleic acid (DNA or RNA) detecting tests. Recently, the first CLIA-waived nucleic acid test platforms have been approved by the US FDA. Thus, a clinic may now have multiple options to obtain a rapid diagnosis—a low-cost RDT that has moderate performance or a higher-cost NAAT that has higher performance. For some patients, in some locations, at certain times of year, the RDT will be sufficient to provide a result with high enough confidence to apply the intervention. For other people, locations, or times of year the RDT performance may be too low to be adequately conclusive, and the more expensive NAAT is more appropriate. These options have only recently become available, and fee-for-service reimbursement in the US has given little incentive to choose less expensive but capable options. Embodiments of the present disclosure can provide the physician evidence that they can use to determine whether to use the more expensive NAAT, or whether the RDT provides adequate performance for a given patient.

Another example of conditions for which multiple tests are available are sexually transmitted diseases such as Gonorrhea or Chlamydia. A test kit for Gonorrhea (or Chlamydia) can include an RDT, a swab and swab buffer tube, a urine cup, and a QR code used to download an app from the manufacturer. A male patient can have the option to collect urine (a simple non-invasive sample with relatively low sensitivity) or to collect a urethral swab (an uncomfortable sample with relatively high sensitivity). After installing the app, the patient could input information requested by the app (male/female, age, burning sensation when urinating, colored discharge, fever, and perhaps more personal information about unsafe sexual practices or history of sexually-transmitted disease). The data can be sent to a server, where it is processed as described above to calculate the post-test probabilities (hypothetical for a positive test and a negative test, since the test has not been performed) for the patient factors, location, and time. The system may return a result that recommends the urethral swab in order to give an actionable post-test probability if the urine test is not predicted to give a conclusive result. When the test is run using the recommended urethral swab, the same or similar calculation is used to determine and report the result as a simple binary result, a binary results with a probability, or a recommended action determined by the device or server. If the same scenario occurred for a male patient with higher pre-test probability (e.g., more symptoms indicating Gonorrhea) the result may be a recommendation that the urine test is sufficient. Often, the sensitivity and specificity of tests like these are also different among men and women due to anatomical differences, and the process described here would account for this effect as well by providing different recommendations for men and women (via their effect on parameters in the calculation). Other examples of choice between sample types include HIV diagnosis using one test (OraSure) with an oral sample (gingival fluid) or a test using a blood sample and tuberculosis diagnosis using a difficult-to-obtain sputum sample (by DNA test or microscopy) or an oral swab (by DNA test) or a urine sample (by a protein (LAM) test or DNA test).

The above descriptions of using embodiments of the present disclosure to perform specific tests relating to flu or gonorrhea are examples provided for the purposes of illustrating some aspects of the disclosed subject matter only, and should not be seen as limiting. In other embodiments, other conditions may be tested for, other types of tests may be performed, other computing devices may be used, and other changes may be made.

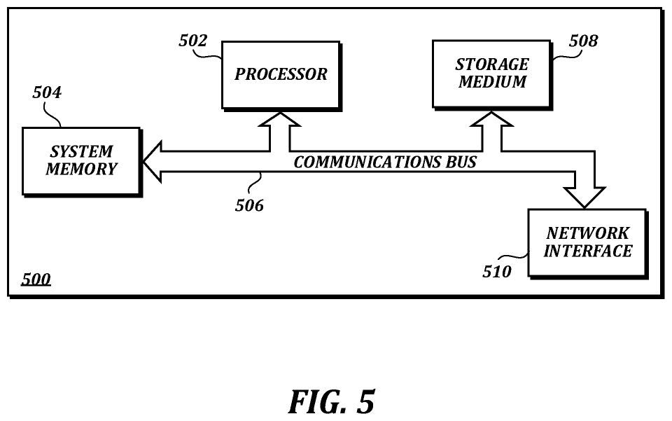

FIG. 5 is a block diagram that illustrates aspects of an exemplary computing device 500 appropriate for use with embodiments of the present disclosure. While FIG. 5 is described with reference to a computing device that is implemented as a device on a network, the description below is applicable to servers, personal computers, mobile phones, smart phones, tablet computers, embedded computing devices, and other devices that may be used to implement portions of embodiments of the present disclosure. Moreover, those of ordinary skill in the art and others will recognize that the computing device 500 may be any one of any number of currently available or yet to be developed devices.

In its most basic configuration, the computing device 500 includes at least one processor 502 and a system memory 504 connected by a communication bus 506. Depending on the exact configuration and type of device, the system memory 504 may be volatile or nonvolatile memory, such as read only memory (“ROM”), random access memory (“RAM”), EEPROM, flash memory, or similar memory technology. Those of ordinary skill in the art and others will recognize that system memory 504 typically stores data and/or program modules that are immediately accessible to and/or currently being operated on by the processor 502. In this regard, the processor 502 may serve as a computational center of the computing device 500 by supporting the execution of instructions.

As further illustrated in FIG. 5, the computing device 500 may include a network interface 510 comprising one or more components for communicating with other devices over a network. Embodiments of the present disclosure may access basic services that utilize the network interface 510 to perform communications using common network protocols. The network interface 510 may also include a wireless network interface configured to communicate via one or more wireless communication protocols, such as Wi-Fi, 2G, 3G, LTE, WiMAX, Bluetooth, and/or the like.

In the exemplary embodiment depicted in FIG. 5, the computing device 500 also includes a storage medium 508. However, services may be accessed using a computing device that does not include means for persisting data to a local storage medium. Therefore, the storage medium 508 depicted in FIG. 5 is represented with a dashed line to indicate that the storage medium 508 is optional. In any event, the storage medium 508 may be volatile or nonvolatile, removable or nonremovable, implemented using any technology capable of storing information such as, but not limited to, a hard drive, solid state drive, flash memory, CD ROM, DVD, or other disk storage, magnetic cassettes, magnetic tape, magnetic disk storage, and/or the like.

As used herein, the term “computer-readable medium” includes volatile and non-volatile and removable and non-removable media implemented in any method or technology capable of storing information, such as computer readable instructions, data structures, program modules, or other data. In this regard, the system memory 504 and storage medium 508 depicted in FIG. 5 are merely examples of computer-readable media. Computer-readable media can be used to store data for use by programs. Accordingly, the terms “electronic spreadsheet,” “grid,” “table,” “cell,” “spreadsheet data,” “sheet data,” “column,” “row,” and others used herein describe display formats and logical inter-relationships for information stored on a computer-readable medium of a computing device 500.

Suitable implementations of computing devices that include a processor 502, system memory 504, communication bus 506, storage medium 508, and network interface 510 are known and commercially available. For ease of illustration and because it is not important for an understanding of the claimed subject matter, FIG. 5 does not show some of the typical components of many computing devices. In this regard, the computing device 500 may include input devices, such as a keyboard, keypad, mouse, microphone, touch input device, touch screen, tablet, and/or the like. Such input devices may be coupled to the computing device 500 by wired or wireless connections including RF, infrared, serial, parallel, Bluetooth, USB, or other suitable connections protocols using wireless or physical connections. Similarly, the computing device 500 may also include output devices such as a display, speakers, printer, etc. Since these devices are well known in the art, they are not illustrated or described further herein.

The specific routines described above in the flowcharts may represent one or more of any number of processing strategies such as event-driven, interrupt-driven, multi-tasking, multi-threading, and the like. As such, various acts or functions illustrated may be performed in the sequence illustrated, in parallel, or in some cases omitted. Likewise, the order of processing is not necessarily required to achieve the features and advantages, but is provided for ease of illustration and description. Although not explicitly illustrated, one or more of the illustrated acts or functions may be repeatedly performed depending on the particular strategy being used. Further, these FIGURES may graphically represent code to be programmed into a computer-readable storage medium associated with a computing device.

While illustrative embodiments have been illustrated and described, it will be appreciated that various changes can be made therein without departing from the spirit and scope of the invention. For example, while healthcare-related techniques were primarily discussed above, some embodiments of the present disclosure may be used for analytic tests unrelated to healthcare, including but not limited to: measurements of structural integrity (defects in airplane parts, bridges, pipelines, storage tanks), detection of pollutants and water quality, presence of radioactive substances, detection and classification of human motion from an accelerometer to detect performance of particular tasks, detection of environmental conditions from deployed sensors (e.g., biothreats, pollution, contamination, water quality), and diagnosis of agricultural conditions (e.g., pest infestation, hydration deficiencies, plant disease, and soil conditions).

1. A method of improving accuracy of a healthcare diagnostic test, the method comprising:
receiving, by a computing device, an individual test result of an analytic test performed on a subject;
determining, by the computing device, a pre-test probability of a condition for the subject;
determining, by the computing device, a likelihood ratio of the analytic test;
determining, by the computing device, augmentation information that affects the likelihood ratio of the analytic test;
generating, by the computing device, an adjusted likelihood ratio using the augmentation information;
determining, by the computing device, a post-test probability of the condition based on the pre-test probability and the adjusted likelihood ratio; and
presenting, by the computing device, the post-test probability on a display device.

2. The method of claim 1, wherein determining the augmentation information includes determining at least one of a subject age, demographics, geographical location, date and time, body temperature, heart rate, respiratory rate, medication status, vaccination status, and expertise level of a test administrator.

3. The method of claim 2, wherein determining the augmentation information includes retrieving factors relevant to the augmentation information from an electronic medical record.

4. The method of claim 1, wherein generating the adjusted likelihood ratio includes:
retrieving, by the computing device from a server, at least one of an updated sensitivity and an updated specificity for the analytic test based on factors relevant to the augmentation information.

5. The method of claim 1, wherein determining the pre-test probability of the condition includes obtaining, by the computing device from a server, at least one of prevalence information for the condition, family illness status, family history, and vaccination status.

6. The method of claim 5, wherein obtaining the prevalence information for the condition includes:
obtaining, by the computing device, a geographical position of the computing device; and
retrieving, by the computing device from a server, prevalence information for a geographic region surrounding the geographical position of the computing device.

7. The method of claim 1, wherein determining the pre-test probability of the condition includes obtaining, by the computing device, a post-test probability of a previous analytic test.

8. The method of claim 1, further comprising:
comparing, by the computing device, the post-test probability to a threshold probability; and
in response to determining that the post-test probability meets the threshold probability, presenting, by the computing device, a recommended intervention for the condition.

9. The method of claim 1, wherein the analytic test is a first analytic test, the method further comprising:
conducting a second analytic test; and
using a test result of the second analytic test to update likelihood ratio information stored on the server for the first analytic test.

10. The method of claim 9, wherein using the test result of the second analytic test to update likelihood ratio information includes:
updating at least one of a sensitivity and a specificity for the first analytic test in association with the augmentation information for the subject.

11-12. (canceled)

13. A method, comprising:
determining, by a computing device, a pre-test probability of a condition for a subject;
determining, by the computing device, a likelihood ratio of a first analytic test;
determining, by the computing device, augmentation information that affects the likelihood ratio of the first analytic test;
generating, by the computing device, an adjusted likelihood ratio using the augmentation information;
determining, by the computing device, a potential post-test probability of the condition based on the pre-test probability, the adjusted likelihood ratio, and an assumption that an individual test result of the first analytic test would indicate the presence of the condition; and
in response to determining that the potential post-test probability is greater than a threshold probability, presenting, by the computing device, an instruction to perform the first analytic test.

14. The method of claim 13, further comprising, in response to determining that the potential post-test probability is not greater than the threshold probability:
determining a second likelihood ratio of a second analytic test;
determining second augmentation information that affects the second likelihood ratio;
generating a second adjusted likelihood ratio using the augmentation information;
determining a second potential post-test probability of the condition based on the pre-test probability, the second adjusted likelihood ratio, and an assumption that an individual test result of the second analytic test would indicate the presence of the condition; and
in response to determining that the second potential post-test probability is greater than the confidence threshold, presenting, by the computing device, an instruction to perform the second analytic test.

15. The method of claim 13, wherein determining the augmentation information includes determining at least one of a subject age, demographics, geographical location, date and time, body temperature, heart rate, respiratory rate, medication status, vaccination status, and expertise level of a test administrator.

16. The method of claim 15, wherein determining the augmentation information includes retrieving factors relevant to the augmentation information from an electronic medical record.

17. The method of claim 13, wherein generating the adjusted likelihood ratio includes:
retrieving, by the computing device from a server, at least one of an updated sensitivity and an updated specificity for the first analytic test based on factors relevant to the augmentation information.

18. The method of claim 13, wherein determining the pre-test probability of the condition includes obtaining, by the computing device from a server, at least one of prevalence information for the condition, family illness status, family history, and vaccination status.

19. The method of claim 18, wherein obtaining the prevalence information for the condition includes:
obtaining, by the computing device, a geographical position of the computing device; and
retrieving, by the computing device from a server, prevalence information for a geographic region surrounding the geographical position of the computing device.

20. The method of claim 13, wherein determining the pre-test probability of the condition includes obtaining, by the computing device, a post-test probability of a previous analytic test.

21-22. (canceled)
